(19) United States
(12) Patent Application Publication
(10) Pub. No.: US 2014/0099330 A1
(43) Pub. Date: Apr. 10, 2014

(54) Method and System for Treating Biological Tissue

(71) Applicant: Robert G. Matheny, Norcross, GA (US)

(72) Inventor: Robert G. Matheny, Norcross, GA (US)

(21) Appl. No.: 14/031,423

(22) Filed: Sep. 19, 2013

Related U.S. Application Data
(60) Provisional application No. 61/710,969, filed on Oct. 8, 2012.

(51) Int. Cl. A61L 27/54; A61K 45/06; A61K 39/395

(52) U.S. Cl. CPC A61L 27/54; A61K 39/39533; A61K 45/06
USPC 424/172.1; 623/23.72

(57) ABSTRACT

A tissue prosthesis comprising a support structure having at least one surface, the support structure comprising a base material, the support structure further including an extracellular matrix (ECM) composition having at least one ECM material from a mammalian tissue source. The tissue prosthesis induces modulated healing of damaged biological tissue when deployed proximate thereto.

### CROSS-REFERENCES TO RELATED APPLICATIONS

This application claims the benefit of U.S. Application Nos. 61/710,969, filed on Oct. 8, 2012.

### FIELD OF THE INVENTION

The present invention relates to implantable biological prostheses for treating biological tissue. More particularly, the present invention relates to non-antigenic, resilient, biocompatible tissue prostheses or grafts that can be engineered into a variety of shapes and used to treat, augment, or replace damaged or diseased biological tissue.

### BACKGROUND OF THE INVENTION

As is well known in the art, tissue prostheses or grafts are often employed to treat or replace damaged or diseased biological tissue. However, despite the growing sophistication of medical technology, the use of grafts to treat or replace damaged biological tissue remains a frequent and serious problem in health care. The problem is often associated with the materials employed to construct the grafts.

As is also well known in the art, the optimal graft material should be chemically inert, non-carcinogenic, capable of resisting mechanical stress, capable of being fabricated in the form required, and sterilizable. Further, the material should be resistant to physical modification by tissue fluids, and not excite an inflammatory reaction, induce a state of allergy or hypersensitivity, or, in some cases, promote visceral adhesions. See, e.g., Jenkins, et al., Surgery, vol. 94(2), pp. 392-398 (1983).

Various materials and/or structures have thus been employed to construct grafts that satisfy the aforementioned optimal characteristics, including tantalum gauze, stainless mesh, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastic®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®).

As discussed in detail below, although some of the noted graft materials satisfy some of the aforementioned optimal characteristics, few, if any, satisfy all of the optimal characteristics.

The major advantages of metallic meshes, e.g., stainless steel meshes, are that they are inert, resistant to infection and can stimulate fibroplasia. Several major disadvantages are fragmentation, which can, and in many instances will, occur after the first year of administration, and the lack of malleability.

Synthetic meshes have the advantage of being easily molded and, except for nylon, retain their tensile strength in or on the body. In European Patent No. 91122196.8 a triple-layer vascular prosthesis is disclosed that utilizes non-resorbable synthetic mesh as the center layer. The synthetic textile mesh layer is used as a central frame to which layers of collagenous fibers are added, resulting in the tri-layered prosthetic device.

There are several drawbacks and disadvantages associated with non-resorbable synthetic mesh. Among the major disadvantages are the lack of inertness, susceptibility to infection, and interference with wound healing.

In contrast to non-resorbable synthetic meshes, absorbable synthetic meshes have the advantage of impermanence at the deployment site, but often have the disadvantage of loss of mechanical strength (as a result of dissolution by the host) prior to adequate cell and tissue ingrowth.

The most widely used graft material for abdominal wall replacement and for reinforcement during hernia repairs is Marlex®, i.e. polypropylene. A major disadvantage associated with polypropylene mesh grafts is that with scar contracture, polypropylene mesh grafts become distorted and separate from surrounding normal tissue.

Gore-Tex®, i.e. polytetrafluoroethylene, is currently believed to be the most chemically inert graft material. However, a major problem associated with the use of polytetrafluoroethylene is that in a contaminated wound it does not allow for any macromolecular drainage, which limits treatment of infections.

Collagen is another commonly employed graft material. Collagen first gained utility as a material for medical use because it was a natural biological graft substitute that was in abundant supply from various animal sources.

The design objectives for the original collagen grafts were the same as for synthetic polymer grafts, i.e. the grafts should persist and essentially act as an inert material. With these objectives in mind, purification and crosslinking methods were developed to enhance mechanical strength and decrease the degradation rate of the collagen.

The crosslinking agents that were originally used included glutaraldehyde, formaldehyde, polyepoxides, diisocyanates and acyl azides. Glutaraldehyde was also used as a sterilizing agent.

A major disadvantage of crosslinking collagen is, however, that it reduces the antigenicity of the material by linking the antigenic epitopes, rendering them either inaccessible to phagocytosis or unrecognizable by the immune system.

Crosslinking collagen will thus, in general, generate collagenous material that resembled a synthetic material more than a natural biological tissue, both mechanically and biologically.

Tissue prostheses or graft material derived from mammalian tissue, i.e. extracellular matrix (ECM), is also often employed to construct tissue prostheses or grafts. Illustrative are the grafts disclosed in U.S. Pat. No. 3,562,820 (tubular, sheet and strip grafts formed from submucosa adhered together by use of a binder paste, such as a collagen fiber paste, or by use of an acid or alkaline medium), and U.S. Pat. No. 4,902,508 (a three layer tissue graft composition derived from small intestine comprising tunica submucosa, the muscularis mucosa, and stratum compactum of the tunica mucosa).

Although a number of the ECM based tissue prostheses or grafts satisfy many of the aforementioned optimal characteristics, efforts continue to develop improved prostheses and/or grafts that can successfully be employed to replace or to facilitate the repair of biological tissue, such as abdominal wall defects and vasculature, whereby the host's own cells can be optimally exploited in the repair process.

It is therefore an object of the present invention to provide tissue prostheses that induce modulated healing; particularly, neovascularization, host tissue proliferation, bioremodeling, and regeneration of tissue and associated structures with site-specific structural and functional properties.

### SUMMARY OF THE INVENTION

The present invention is directed to non-antigenic, resilient, bioremodelable, biocompatible tissue prostheses that can be engineered into a variety of shapes and used to repair, augment, or replace mammalian tissues and organs.

In a preferred embodiment, the tissue prostheses include a support structure and an extracellular matrix (ECM) composition. According to the invention, the support structure can comprise various conventional metals, and synthetic and natural materials, including, without limitation, tantalum gauze, stainless mesh, Dacron®, Orlon®, Fortisan®, nylon, knitted polypropylene (e.g., Marlex®), microporous expanded-polytetrafluoroethylene (e.g., Gore-Tex®), Dacron® reinforced silicone rubber (e.g., Silastlc®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®).

In some embodiments of the invention, the support structure is coated with an ECM composition. In some embodiments, the support structure is impregnated with an ECM composition.

In a preferred embodiment of the invention, the ECM compositions include at least one ECM material. According to the invention, the ECM material can be derived from various mammalian tissue sources, including the small intestine, large intestine, stomach, lung, liver, kidney, pancreas, placenta, heart, bladder, prostate, mesothelium, tissue surrounding growing enamel, tissue surrounding growing bone, and any fetal tissue from any mammalian organ, and methods for preparing same.

In some embodiments, the ECM compositions further include one or more additional biologically active components to facilitate the treatment of damaged tissue and/or the tissue regenerative process.

In some embodiments, the ECM compositions thus include at least one pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent specifically comprises an anti-inflammatory agent or composition.

In some embodiments of the invention, the biologically active component comprises a statin. According to the invention, suitable statins include, without limitation, atorvastatin, cerivastatin, fluvastatin, lovastatin, mevastatin, pitavastatin, pravastatin, rosuvastatin, and simvastatin.

In some embodiments of the invention, the biologically active component comprises chitin, chitosan or a derivative thereof.

In some embodiments of the invention, the biologically active component comprises a cell.

In some embodiments of the invention, the biologically active component comprises a protein.

According to the invention, upon deployment of a tissue prosthesis of the invention, modulated healing and regeneration of tissue structures with site-specific structural and functional properties are effectuated.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of a tissue prosthesis, in accordance with the invention; and

FIG. 2 is a side elevation, partial sectional view of the tissue prosthesis shown in FIG. 1, in accordance with the invention.

### DETAILED DESCRIPTION OF THE PREFERRED EMBODIMENT

Before describing the present invention in detail, it is to be understood that this invention is not limited to particularly exemplified apparatus, systems, compositions or methods as such may, of course, vary. Thus, although a number of systems, compositions and methods similar or equivalent to those described herein can be used in the practice of the present invention, the preferred systems, compositions and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an anti-inflammatory” includes two or more such agents and the like.

### DEFINITIONS

The terms “tissue prosthesis” and “graft” are used interchangeably herein, and mean and include a prosthetic device that is configured to be placed on or over biological tissue, or in a vascular structure, e.g. a stent, to treat or replace damaged or diseased biological tissue.

The terms “damaged tissue” and “diseased tissue” are used interchangeably herein, and mean and include any area of abnormal biological tissue caused by a disease, disorder, injury or damage, including damage to the epicardium, endocardium and/or myocardium. Non-limiting examples of causes of cardiovascular tissue damage include acute or chronic stress (systemic hypertension, pulmonary hypertension, valve dysfunction, etc.), coronary artery disease, ischemia or infarction, inflammatory disease and cardiomyopathies.

The terms “prevent” and “preventing” are used interchangeably herein, and mean and include reducing the frequency or severity of a disease, condition or disorder. The term does not require an absolute preclusion of the disease, condition or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder.

The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “extracellular matrix”, “extracellular matrix material” and “ECM material” are used interchangeably herein, and mean a collagen-rich substance that is found in between cells in animal tissue and serves as a structural element in tissues. It typically comprises a complex mixture of polysaccharides and proteins secreted by cells. The extracellular matrix can be isolated and treated in a variety of ways. Extracellular matrix material (ECM) can be isolated from small intestine submucosa, stomach submucosa, urinary bladder submucosa, tissue mucosa, dura mater, liver basement membrane, pericardium or other tissues. Following isolation and treatment, it is commonly referred to as extracellular matrix or ECM material.

The terms “pharmacological agent”, “pharmaceutical agent”, “agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “pharmaceutical agent”, “agent”, “active agent”, “drug” and “active agent formulation” thus mean and include, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antitluombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues. Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, connethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “chitosan”, as used herein, means and includes the family of linear polysaccharides consisting of varying amounts of β(1→4) linked residues of N-acetyl-2 amino-2-deoxy-D-glucose and 2-amino-2-deoxy-Dglucose residues, and all derivatives thereof.

The terms “active agent formulation”, “pharmacological agent formulation” and “agent formulation”, are also used interchangeably herein, and mean and include an active agent (and chitosan) optionally in combination with one or more pharmaceutically acceptable carriers and/or additional inert ingredients. According to the invention, the formulations can be either in solution or in suspension in the carrier.

The term “pharmacological composition”, as used herein, means and includes a composition comprising a “pharmacological agent” and/or an “extracellular matrix material” and/or a “pharmacological agent formulation” and/or any additional agent or component identified herein.

The term “therapeutically effective”, as used herein, means that the amount of the “pharmacological composition” and/or “pharmacological agent” and/or “active agent formulation” administered is of sufficient quantity to ameliorate one or more causes, symptoms, or sequalae of a disease or disorder. Such amelioration only requires a reduction or alteration, not necessarily elimination, of the cause, symptom, or sequalae of a disease or disorder.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As will readily be appreciated by one having ordinary skill in the art, the present invention substantially reduces or eliminates the disadvantages and drawbacks associated with prior art methods of treating damaged or diseased biological tissue.

In overview, the present disclosure is directed to non-antigenic, resilient, bioremodelable, biocompatible tissue prostheses or grafts that can be engineered into a variety of shapes and used to repair, augment, or replace mammalian tissues and organs.

In a preferred embodiment, the tissue prostheses include a support structure and an extracellular matrix (ECM) composition. According to the invention, the support structure can comprise various conventional metals, and synthetic and natural materials, including, without limitation, tantalum gauze, stainless mesh, Dacron®, Orlon®, Fortisan®, nylon, knitted polypropylene (e.g., Marlex®), microporous expanded-polytetrafluoroethylene (Gore-Tex®), Dacron reinforced silicone rubber (e.g., Silastic®), polyglactin 910 (e.g., Vicryl®), polyester (e.g., Mersilene®), polyglycolic acid (Dexon®) processed sheep dermal collagen, crosslinked bovine pericardium (e.g., Peri-Guard®), and preserved human dura (e.g., Lyodura®).

In some embodiments of the invention, the support structure is coated with an ECM composition of the invention. In some embodiments, the support structure is impregnated with an ECM composition of the invention.

According to the invention, upon administration of a tissue prosthesis of the invention to (or proximate to) damaged or diseased biological tissue, modulated healing, including regeneration of tissue structures with site-specific structural and functional properties, is effectuated.

The phrase “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the ECM compositions of the invention are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase.

In some embodiments, “modulated healing” refers to the ability of an ECM composition to alter a substantial inflammatory phase (e.g., platelet or fibrin deposition) at the beginning of the tissue healing process. As used herein, the phrase “alter a substantial inflammatory phase” refers to the ability of an ECM composition to substantially reduce the inflammatory response at an injury site.

In such an instance, a minor amount of inflammation may ensue in response to tissue injury, but this level of inflammation response, e.g., platelet and/or fibrin deposition, is substantially reduced when compared to inflammation that takes place in the absence of an ECM composition of the invention.

For example, the ECM compositions discussed herein have been shown experimentally to delay or alter the inflammatory response associated with damaged tissue, as well as excessive formation of connective fibrous tissue following tissue damage or injury. The ECM compositions have also been shown experimentally to delay or reduce fibrin deposition and platelet attachment to a blood contact surface following tissue damage.

In some embodiments of the invention, “modulated healing” refers to the ability of an ECM composition of the invention to induce host tissue proliferation, bioremodeling, including neovascularization, e.g., vasculogenesis, angiogenesis, and intussusception, and regeneration of tissue structures with site-specific structural and functional properties.

In a preferred embodiment of the invention, the ECM compositions include at least one extracellular matrix (hereinafter “ECM material”). According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

As is well known in the art, the urinary bladder submucosa is an extracellular matrix that has the tunica mucosa (which includes the transitional epithelial layer and the tunica propria), a submucosal layer, three layers of muscularis, and the adventitia (a loose connective tissue layer). This general configuration is true also for small intestine submucosa (SIS) and stomach submucosa (SS).

Other tissues, such as the liver and pancreas have ECM material called basement membrane. Basement membrane generally does not demonstrate the kind of tensile strength found in submucosa. However, other useful properties may be opportunistically employed from the ECM material of such tissues as the liver, pancreas, placenta and lung tissues; all of which have either a basement membrane or interstitial membrane (as with the lung). For example, pancreatic extracellular membrane supports beta islet cells that are critical to pancreatic function. Also, for example, the liver is one tissue known to be able to regenerate itself and therefore special qualities may be present in the liver basement membrane that help facilitate that process. The ECM material surrounding developing tooth enamel and developing bone also have particular advantages over other matrices in that they support the growth and differentiation of the hard tissues of bone and enamel.

According to the invention, the ECM material can be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the ECM or matrix composition from any given source will contain the active extracellular matrix portions that support cell development and differentiation and tissue regeneration.

For purposes of this invention, the ECM material from any of the mammalian tissue consists of several basically inseparable layers broadly termed ECM material. For example, where it is thought that separating a basement membrane from the submucosa is considered to be very difficult, if not impossible, because the layers are thin and it is not possible to delaminate them from each other, the ECM material from that particular layer will probably necessarily contain some basement membrane with the submucosa.

According to the invention, the ECM compositions of the invention can also comprise ECM material from two or more mammalian sources. Thus, for example, the composition can comprise ECM material combinations from such sources as, for example, but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart ECM material, dermal matrix, and, in general, ECM material from any mammalian fetal tissue. The ECM material sources can also comprise different mammalian animals or an entirely different species of mammals.

The ECM composition can thus comprise ECM material from three mammalian tissue sources, four mammalian tissue sources, five mammalian tissue sources, six mammalian tissue sources, and conceivably up to ten or more tissue sources. The tissue sources can be from the same mammal (for example the same cow, the same pig, the same rodent, the same human, etc.), the same species of mammal (e.g. cow, pig, rodent, human), or different mammalian animals, but the same species, (e.g. cow 1 and cow 2, or pig 1 and pig 2), or different species of mammals (for example liver matrix from a pig, small intestine submucosa from a cow, and urinary bladder submucosa from a dog, all mixed together in the composition).

According to the invention, the ECM material can comprise mixed solid particulates. The ECM material can also be formed into a particulate and fluidized, as described in U.S. Pat. Nos. 5,275,826, 6,579,538 and 6,933,326, to form a mixed emulsion, mixed gel or mixed paste.

According to the invention, the liquid or semi-solid components of the ECM compositions (i.e. gels, emulsions or pastes) can comprise various concentrations. Preferably, the concentration of the liquid or semi-solid components of the ECM compositions are in the range of about 0.001 mg/ml to about 200 mg/ml. Suitable concentration ranges thus include, without limitation: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml and about 40 mg/ml. to about 42 mg/ml.

The noted concentration ranges are, however, merely exemplary and not intended to be exhaustive or limiting. It is understood that any value within any of the listed ranges is deemed a reasonable and useful value for a concentration of a liquid or semi-solid component of an ECM composition.

According to the invention, the dry particulate or reconstituted particulate that forms a gel emulsion or paste of the two ECM materials can also be mixed together in various proportions. For example, the particulates can comprise 50% of small intestine submucosa mixed with 50% of pancreatic basement membrane. The mixture can then similarly be fluidized by hydrating in a suitable buffer, such as saline.

According to the invention, the ECM compositions of the invention can further include one or more additional bioactive agents or components to aid in the treatment of damaged tissue and/or facilitate the tissue regenerative process.

In some embodiments, the ECM compositions of the invention thus include at least one pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

Suitable pharmacological agents and/or compositions thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oxytetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, Anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

According to the invention, the amount of a pharmacological agent added to an ECM composition of the invention will, of course, vary from agent to agent. For example, in one embodiment, wherein the pharmacological agent comprises dicloflenac (Voltaren®), the amount of dicloflenac included in the ECM composition is preferably in the range of 10 μg-75 mg.

In some embodiments of the invention, the pharmacological agent specifically comprises an anti-inflammatory agent. According to the invention, suitable anti-inflammatory agents include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sennetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

According to the invention, the amount of an anti-inflammatory added to an ECM composition of the invention can similarly vary from anti-inflammatory to anti-inflammatory. For example, in one embodiment of the invention, wherein the pharmacological agent comprises ibuprofen (Advil®), the amount of ibuprofen included in the ECM composition is preferably in the range of 100 μg-200 mg.

In some embodiments of the invention, the pharmacological agent comprises a statin, i.e. a HMG-CoA reductase inhibitor. According to the invention, suitable statins include, without limitation, atorvastatin (LIPITOR®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. Several significant properties and beneficial actions resulting therefrom are discussed in detail below. Additional properties and beneficial actions are set forth in Co-Pending application Ser. No. 13/373,569; which is incorporated by reference herein in its entirety.

### Anti-Inflammatory Properties/Actions

Statins have numerous favorable effects on vascular wall cells and the cardiovascular system. One specific example is that statins facilitate the reduction of the G-Protein-Coupled Receptor, thromboxane A2 (TXA₂), which lowers the platelet activation and aggregation, and augmentation of adhesion molecules and chemokines. Statins further impact vascular wall cells and the cardiovascular system by blocking ras homilog gene family, member A (RhoA) activation. Blocking RhoA activation further impacts numerous systems, such as macrophage growth, tissue plasminogen activators (t-PA), plasminogen activator inhibitor type 1 (PAI-1), smooth muscle cell (SMC) proliferation, nitric oxide (NO) production, endothelins, and angiotensin receptors.

Macrophage growth reduced by blocking RhoA activation results in the reduction of matrix metalloprotinases (MMPs) and tissue factors (TF). Lowered MMPs also results in a lowered presence of thrombi, as the MMPs attach to ECM present in thrombi or damaged ECM at wound sites.

### Fibrinolysis Properties/Actions

Blocking RhoA activation also affects the presence of tissue plasminogen activators (t-PA) and plasminogen activator inhibitor type 1 (PAI-1), which is the principal inhibitor of fibrinolysis. With t-PA presence raised and PAI-1 diminished from the blocking of RhoA activation induced by statins, a reduced thrombotic effect is realized due to reduced opportunity for fibrin to form the polymeric mesh of a hemostatic plug.

### NO Regulation Properties/Actions

Blocking RhoA activation also affects the presence of Nitric Oxide (NO) in the cardiovascular system. NO contributes to vessel homeostasis by inhibiting vascular smooth muscle contraction and growth, platelet aggregation, and leukocyte adhesion to the endothelium.

### RhoA Activation Blocking Properties/Actions

The administration of statins can also enhance the presence of endothelins and angiotensin receptors. Endothelins and angiotensin receptors can also be affected by the subsequent blocking of RhoA activation associated with statin administration.

There are three isoforms of endothelins; ET-1, ET-2, and ET-3, with ET-1 being the isoform primarily affected by statins and RhoA activation blocking. Secretion of ET-1 from the endothelium signals vasoconstriction and influences local cellular growth and survival.

Angiotensin receptors are protein coupled receptors that are responsible for the signal transduction of the vasoconstricting stimulus of the main effector hormone angiotensin II. Angiotensin Receptor II Type I (AT-1) is the angiotensin receptor primarily affected by statin administration and RhoA activation blocking. AT-1 mediates vasocontraction, cardiac hypertrophy, vascular smooth muscle cell proliferation, inter alia.

### C-Reactive Protein Reduction Properties/Actions

C-Reactive Proteins (CRP) are also reduced by statins. CRPs are found in the blood; the levels of which deviate in response to differing levels of inflammation.

### Adhesion Molecule Reduction Properties/Actions

Statins also reduce the presence of adhesion molecules on the endothelium. Adhesion molecules are proteins that are located on the cell surface and are involved with inflammation and thrombin formation in vascular endothelial cells.

### Rac-1 Reduction Properties/Actions

The expression of Rac-1 is also reduced by statins. Rac-1 is a protein found in human cells, which plays a central role in endothelial cell migration, tubulogenesis, adhesion, and permeability. The decrease in the presence of Rac-1 also results in the decrease of reactive oxygen species (ROS).

According to the invention, the ECM support member (or material) can include 10 mg or greater of a statin to achieve a higher concentration of the statin within a desired tissue, or 10 ug or less to achieve a lower concentration of the statin within a desired tissue.

According to the invention, the amount of a statin added to a pharmacological composition of the invention is preferably less than 20 mg, more preferably, less than approximately 10 mg.

In some embodiments of the invention, the ECM material includes 100 ug-5 mg of a statin. In some embodiments of the invention, the ECM material includes 500 ug-2 mg of a statin.

In some embodiments of the invention, the ECM support member (or material) includes chitosan or a derivative thereof. As also set forth in detail in Co-Pending application Ser. No. 13/573,569, chitosan also exhibits numerous beneficial properties that provide several beneficial biochemical actions or activities.

According to the invention, the amount of chitosan added to a pharmacological composition of the invention is preferably less than 50 ml, more preferably, less than approximately 20 ml.

In some embodiments of the invention, the chitosan is incorporated in a polymeric network, such as disclosed in U.S. Pub. Nos. 2008/0254104 and 2009/0062849, which are incorporated herein in their entirety.

In some embodiments of the invention, the bioactive agent comprises a cell. According to the invention, the cell can comprise, without limitation, a stem cell, such as, for example, a human embryonic stem cell, fetal cell, fetal cardiomyocyte, myofibroblast, mesenchymal stem cell, autotransplanted expanded cardiomyocyte, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, myofibroblast, osteoblast, chondrocyte, exogenous cell, endogenous cell, stem cell, hematopoetic stem cell, pluripotent stem cell, bone marrow-derived progenitor cell, progenitor cell, myocardial cell, skeletal cell, undifferentiated cell, multi-potent progenitor cell, unipotent progenitor cell, monocyte, cardiomyocyte, cardiac myoblast, skeletal myoblast, macrophage, capillary endothelial cell, xenogenic cell, and allogenic cell.

In some embodiments of the invention, the bioactive agent comprises a protein. According to the invention, the protein can comprise, without limitation, a growth factor, collagen, proteoglycan, glycosaminoglycan (GAG) chain, glycoprotein, cytokine, cell-surface associated protein, cell adhesion molecule (CAM), angiogenic growth factor, endothelial ligand, matrikine, matrix metalloprotease, cadherin, immunoglobin, fibril collagen, non-fibrillar collagen, basement membrane collagen, multiplexin, small-leucine rich proteoglycan, decorin, biglycan, fibromodulin, keratocan, lumican, epiphycan, heparan sulfate proteoglycan, perlecan, agrin, testican, syndecan, glypican, serglycin, selectin, lectican, aggrecan, versican, nuerocan, brevican, cytoplasmic domain-44 (CD44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

In some embodiments of the invention, the ECM compositions specifically include a statin and chitosan. It has been found that the synergistic actions exhibited by the combination of a statin and chitosan significantly enhance the inducement of neovascularization, host tissue proliferation, bioremodeling, and regeneration of new tissue and associated structures (with site-specific structural and functional properties) when administered to damaged or diseased biological tissue.

According to the invention, the bioactive agents referenced above can comprise any form. In some embodiments of the invention, the bioactive component or components, e.g. simvastatin and/or chitosan, comprise microcapsules that provide delayed delivery of the agent contained therein.

As indicated above, in some embodiments of the invention, one or more ECM compositions of the invention are coated on a support structure to form a tissue prosthesis or graft of the invention. In some embodiments of the invention, one or more ECM compositions of the invention are incorporated into a support structure to form a tissue prosthesis of the invention. In some embodiments of the invention, one or more ECM compositions of the invention are coated on and incorporated into a support structure to form a tissue prosthesis.

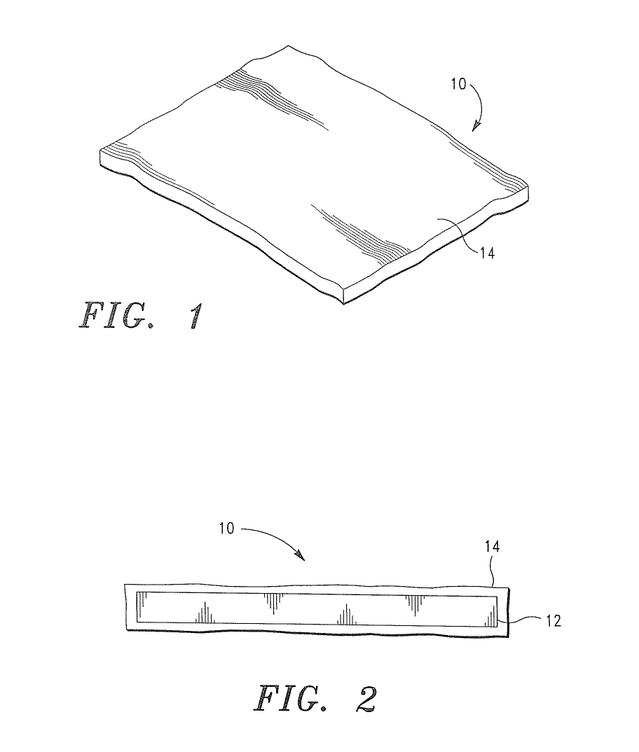

Referring now to FIGS. 1 and 2 there is shown a tissue prosthesis of the invention 10 having a support structure 12 with an ECM composition coating 14 disposed thereon. As indicated above, the support structure can comprise various conventional metals, and synthetic and natural materials, including, without limitation, tantalum gauze, stainless mesh, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastic®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®). The support structure 12 can also comprise an ECM based material.

According to the invention, various conventional means can be employed to form the tissue prosthesis 10, including spray coating, dipping, etc.

To enhance the engagement of a tissue prosthesis of the invention to biological tissue, in some envisioned embodiments, the support structure 12 can also comprise a microneedle support structure, such as disclosed in U.S. application Ser. No. 13/686,131, which is expressly incorporated herein in its entirety.

As will readily be appreciated by one having ordinary skill in the art, the present invention provides numerous advantages compared to prior art methods and systems for treating damaged cardiac tissue. Among the advantages are the following:
The provision of tissue prostheses, which, when delivered to damaged or diseased biological tissue, induce modulated healing, including regeneration of tissue structures with site-specific structural and functional properties.
The provision of extracellular matrix (ECM) compositions which, when employed with a base prosthesis support structure, induce host tissue proliferation, bioremodeling, and regeneration of tissue structures with site-specific structural and functional properties.
The provision of improved methods and apparatus for administering pharmacological compositions; particularly, ECM compositions directly to damaged or diseased biological tissue.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A tissue prosthesis, comprising:
a support structure having at least one surface, said support structure comprising a base material,
said support structure further including an extracellular matrix (ECM) composition, said ECM composition including at least one ECM material from a mammalian tissue source,
wherein, when said tissue prosthesis is deployed proximate damaged biological tissue, said prosthesis induces modulated healing of said damaged tissue.

2. The tissue prosthesis of claim 1, wherein said support structure comprises a synthetic material selected from the group consisting of Dacron®, Orlon®, Fortisan®, nylon, knitted polypropylene, microporous expanded-polytetrafluoroethylene, Dacron® reinforced silicone rubber, and polyester.

3. The tissue prosthesis of claim 1, wherein said support structure comprises a natural material selected from the group consisting of processed sheep dermal collagen, crosslinked bovine pericardium, and preserved human dura.

4. The tissue prosthesis of claim 1, wherein said support structure comprises a metal structure selected from the group consisting of tantalum gauze and stainless mesh.

5. The tissue prosthesis of claim 1, wherein said ECM material is selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, omamentum extracellular matrix, heart extracellular matrix and lung extracellular matrix.

6. The tissue prosthesis of claim 1, wherein said ECM material comprises a decellularized ECM material.

7. The tissue prosthesis of claim 1, wherein said ECM material includes at least one supplemental biologically active agent.

8. The tissue prosthesis of claim 7, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

9. The tissue prosthesis of claim 7, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

10. The tissue prosthesis of claim 7, wherein said biologically active agent comprises an active agent selected from the group consisting of a collagen (types I-V), proteoglycans, glycosaminoglycans (GAGS), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, (inulin II, integrins, transmembrane molecules, thrombospondins, ostepontins, and angiotensin converting enzymes (ACE).

11. The tissue prosthesis of claim 7, wherein said biologically active agent comprises a pharmacological agent.

12. The tissue prosthesis of claim 11, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

13. The tissue prosthesis of claim 11, wherein said pharmacological agent comprises a HMG-CoA reductase inhibitor.

14. The tissue prosthesis of claim 13, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

15. The tissue prosthesis of claim 1, wherein said ECM composition is deposited on said support structure surface.

16. The tissue prosthesis of claim 1, wherein said ECM composition is impregnated in said support structure base material.
